(19) United States
(12) Patent Application Publication
(10) Pub. No.: US 2009/0005650 A1
(43) Pub. Date: Jan. 1, 2009

(54) METHOD AND APPARATUS FOR IMPLEMENTING DIGITAL VIDEO MODELING TO GENERATE A PATIENT RISK ASSESSMENT MODEL

(76) Inventors: Robert Lee Angell, Salt Lake City, UT (US); James R. Kraemer, Santa Fe, NM (US)

Correspondence Address: DUKE W. YEE, YEE AND ASSOCIATES, P.C., P.O. BOX 802333, DALLAS, TX, 75380, US

(21) Appl. No.: 11/771,884

(22) Filed: Jun. 29, 2007

(51) Int. Cl. A61B 5/00; G06Q 50/00

(52) U.S. Cl. 600/300; 705/3

(57) ABSTRACT

A computer implemented method, apparatus, and computer program product for generating a risk assessment model for an assessment of a patient in a healthcare facility. The process retrieves event data for the patient, wherein the event data is derived from video data, and wherein the event data further comprises metadata describing events affecting the patient in a medical care facility, and parses the event data to form assessment data. The process then generates the risk assessment model using the assessment data.

### CROSS-REFERENCE TO RELATED APPLICATIONS

The present invention is related to the application entitled Intelligent Surveillance System and Method for Integrated Event Based Surveillance, application Ser. No. 11/455,251 (filed Jun. 16, 2006), assigned to a common assignee, and which is incorporated herein by reference.

### BACKGROUND OF THE INVENTION

1. Field of the Invention

The present invention relates generally to an improved data processing system, and in particular, to a computer implemented method and apparatus for processing video and audio data. Still more particularly, the present invention relates to a computer implemented method, apparatus, and computer usable program product for utilizing digital video modeling to generate a patient risk assessment model for identifying morbidity and mortality based on events occurring in a medical care facility.

2. Description of the Related Art

Medical care facilities are hectic environments filled with patients suffering from a variety of medical conditions. The number of patients being treated in medical care facilities is increasing as a result of a number of different factors. The factors include, for example, a growing number of uninsured people, an aging population contracting age-related illnesses and chronic health conditions, development of new strains of bacteria and viruses, and an increasing number of elective surgeries. These patients are treated and tended to by doctors, nurses, assistants, technicians, and other medical care workers. However, the shortage of medical care workers in medical care facilities often means that such facilities are understaffed and overcrowded.

To treat all the patients in a medical care facility, medical care workers are often required to work longer hours and tend to more patients than medical care workers in the past. The overworked medical care workers are often tired, stressed, and under pressure. As a result, the quality of patient care diminishes and careless errors occur. The careless errors may exacerbate an existing medical condition of a patient, create a new medical condition, or may result in a serious condition being overlooked.

Thus, a patient's condition may also be affected by actions or omissions by medical care workers, or by the occurrence of events in a medical care facility. These actions, events, or omissions may be unplanned or inadvertent and thus undocumented in a patient's medical chart. Consequently, the chart is of no help to plan a course of treatment or determine the cause of a patient's condition. In addition, medical charts may be incomplete, inaccurate, or illegible, and thus, useless in the evaluation of a patient's condition and the formulation of a treatment strategy.

### SUMMARY OF THE INVENTION

The illustrative embodiments described herein provide a computer implemented method, apparatus, and computer usable program product for generating a risk assessment model for an assessment of a patient. The process retrieves event data for the patient, wherein the event data is derived from video data, and wherein the event data further comprises metadata describing events affecting the patient in a medical care facility, and parses the event data to form assessment data. The process then generates the risk assessment model using the assessment data.

### BRIEF DESCRIPTION OF THE DRAWINGS

The novel features believed characteristic of the invention are set forth in the appended claims. The invention itself, however, as well as a preferred mode of use, further objectives and advantages thereof, will best be understood by reference to the following detailed description of an illustrative embodiment when read in conjunction with the accompanying drawings, wherein:

FIG. 1 is a pictorial representation of a network data processing system in which illustrative embodiments may be implemented;

FIG. 2 is a simplified block diagram of a medical care facility in which a set of sensors may be deployed;

FIG. 3 is a block diagram of a data processing system in which the illustrative embodiments may be implemented;

FIG. 4 is a diagram of a smart detection system for generating event data in accordance with a preferred embodiment of the present invention;

FIG. 5 is a block diagram of a data processing system for analyzing event data to generate a patient risk assessment model in accordance with an illustrative embodiment;

FIG. 6 is a block diagram of a unifying data model for processing event data in accordance with an illustrative embodiment;

FIG. 7 is a block diagram of a data flow through a smart detection system in accordance with an illustrative embodiment; and

FIG. 8 is a flowchart of a process for generating a patient risk assessment model in accordance with an illustrative embodiment.

### DETAILED DESCRIPTION OF THE PREFERRED EMBODIMENT

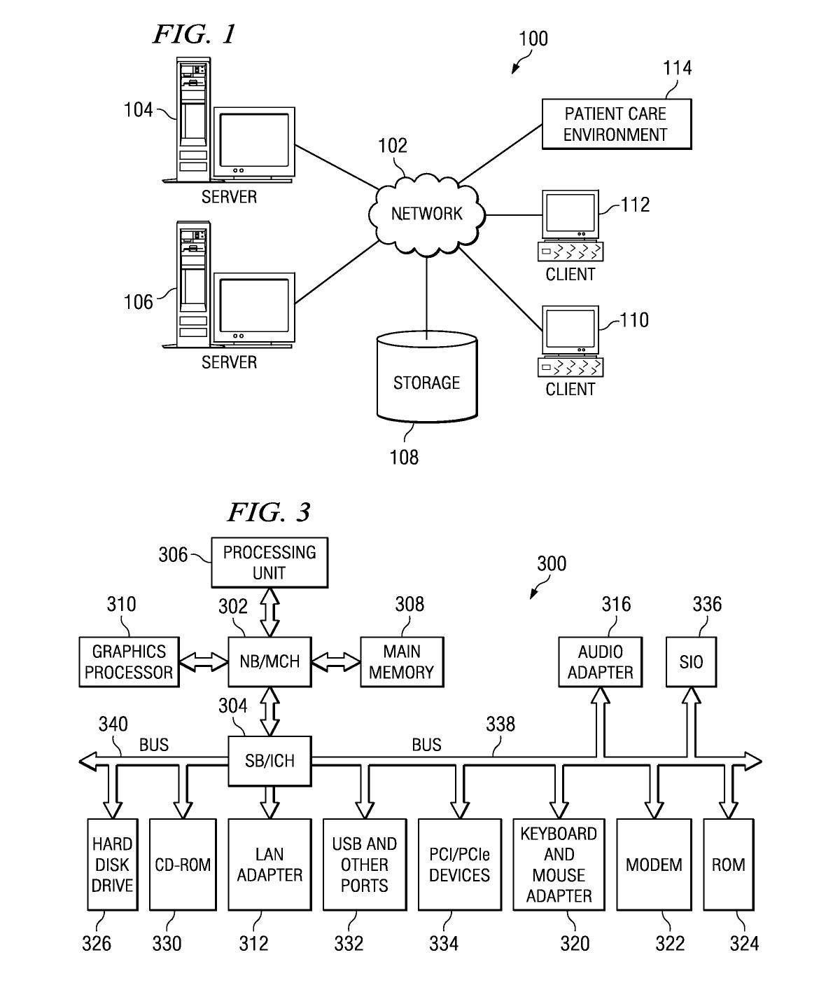

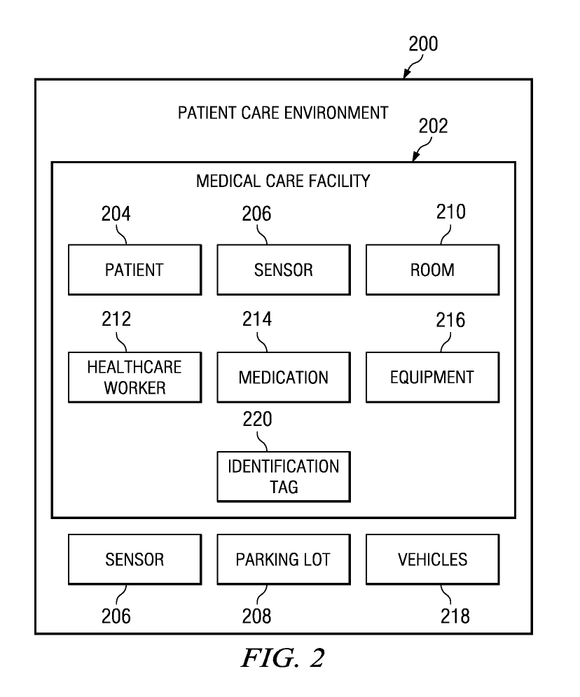

With reference now to the figures, and in particular, with reference to FIGS. 1-2, exemplary diagrams of data processing environments are provided in which illustrative embodiments may be implemented. It should be appreciated that FIGS. 1-2 are only exemplary and are not intended to assert or imply any limitation with regard to the environments in which different embodiments may be implemented. Many modifications to the depicted environments may be made.

FIG. 1 depicts a pictorial representation of a network of data processing systems in which illustrative embodiments may be implemented. Network data processing system 100 is a network of computers in which the illustrative embodiments may be implemented. Network data processing system 100 contains network 102, which is the medium used to provide communications links between various devices and computers connected together within network data processing system 100. Network 102 may include connections such as wire, wireless communication links, or fiber optic cables.

In the depicted example, server 104 and server 106 connect to network 102 along with storage 108. In addition, clients 110 and 112 connect to network 102. Clients 110 and 112 may be, for example, personal computers or network computers. In the depicted example, server 104 provides data, such as boot files, operating system images, and applications to clients 110 and 112. Clients 110 and 112 are clients to server 104 in this example. Network data processing system 100 may include additional servers, clients, and other computing devices not shown.

In the depicted example, network data processing system 100 is the Internet with network 102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, network data processing system 100 may also be implemented as a number of different types of networks, such as for example, an intranet, a local area network (LAN), or a wide area network (WAN). FIG. 1 is intended as an example, and not as an architectural limitation for the different illustrative embodiments.

Networking data processing system 100 also includes patient care environment 114. Patient care environment 114 is an environment in which patients receive healthcare services. Healthcare services are services that directly or indirectly affect a patient. For example, healthcare services that directly affect a patient may include changing a patient's dressings, helping a patient to the bathroom, feeding a patient, monitoring the patient's vital statistics, or administering medication to the patient. Healthcare services may also include events that indirectly affect a patient, such as sterilizing equipment, cleaning rooms, filling out paperwork, delivering supplies to the various supply rooms, and transmitting information from one healthcare worker to another.

Patient care environment 114 may include one or more facilities, buildings, or other structures, such as parking lots, for use in the provision of healthcare services. A parking lot may include an open air parking lot, an underground parking garage, an above ground parking garage, an automated parking garage, and/or any other area designated for storing vehicles. In addition, patient care environment 114 may include any type of equipment, tool, vehicle, or medical care worker capable of providing healthcare services.

FIG. 2 depicts a simplified block diagram of a patient care environment in which illustrative embodiments may be implemented. In this illustrative embodiment in FIG. 2, patient care environment 200 is a patient care environment such as patient care environment 114 in FIG. 1.

Patient care environment 200 includes medical care facility 202. Medical care facility 202 is a facility in which healthcare services are provided to patient 204. Patient 204 is one or more persons seeking healthcare services at medical care facility 202. Medical care facility 202 may be a hospital, a nursing home, a rehabilitation facility, an outpatient clinic, an emergency room, or a personal residence. In alternate embodiments, where patient 204 includes animals, medical care facility 202 may be a veterinary clinic, a ranch, or a zoo. Patient care environment 200 includes one or more sensors for gathering event data at patient care environment 200. Event data is data and metadata describing actions and events that occur in a patient care environment, such as patient care environment 200. In particular, event data includes audio and video data collected by from video cameras deployed throughout patient care environment 200. For example, event data could describe a manner in which a doctor operates on a patient, a path that a nurse takes to arrive at a patient's room, the various locations that a healthcare worker visits during the course of a day, the number of motions that a nurse performs to change a patient's dressings, an amount of time that elapses after a patient has entered an emergency room or pressed a call button, an amount of time that elapsed before a doctor's order was filled, a length of time that tools were sterilized in an autoclave, the medications a nurse administers to a patient, a patient's symptoms, pedestrian traffic throughout the medical care facility, a time that an ambulance brought a patient to the emergency room, or any other action or event that may occur in a patient care environment, such as patient care environment 200.

To gather event data, patient care environment 200 includes sensor 206. Sensor 206 is a set of one or more sensors deployed at patient care environment 200 for monitoring a location, an object, or a person. Sensor 206 may be located internally and/or externally to medical care facility 202. For example, sensor 206 may be mounted to light poles in parking lot 208, above a doorway or entrance to medical care facility 202, or attached to the roof of medical care facility 202. In addition, sensor 206 may be placed in a hallway within medical care facility 202, or mounted within room 210.

Room 210 is one or more rooms that may be found in a medical care facility, such as medical care facility 202. For example, room 210 may be a patient recovery room, an intensive care unit, a nurse's station, an employee lounge, a supply room, a bathroom, an elevator, an emergency room, an imaging room, a pathology lab, a radiology lab, or a cafeteria. In addition, one or more persons or objects may be located in room 210. For example, where room 210 is a patient recovery room, then room 210 may contain patient 204, and optionally healthcare worker 212 assisting patient 204.

Where room 210 is a pharmacy, then room 210 may be stocked with medication 214. Medication 214 is medicine administered to patient 204 for treatment of medical conditions. Medication 214 may be, for example, anesthetics, ointments, antibiotics, pills, or any other form of drug or medication that may be provided to patient 204.

Additionally, room 210 may contain equipment 216. Equipment 216 is any type of equipment found in a medical care facility for use in providing healthcare services to a patient. Equipment 216 may include, for example, x-ray machines, MRI machines, scales, monitors, syringes, scalpels, blankets, or any other tool or piece of equipment found in a medical care facility.

When deployed internally to medical care facility 202, sensor 206 is operable to collect event data relating to the provision of healthcare services to patient 204 by healthcare worker 212 within medical care facility 202. When deployed externally to medical care facility 202, sensor 206 may be used to monitor locations, objects, and people in the areas external to medical care facility 202. For example, sensor 206 may monitor parking lot 208 and vehicles 218 for gathering event data that may be relevant to the provision of healthcare services. For example, vehicles 218 may be an ambulance that delivered patient 204 to medical care facility 202. Thus, sensor 206 monitoring vehicles 218 may capture event data describing a time when patient 204 arrived at medical care facility 202 and a condition of patient 204 upon arrival. Further, where sensor 206 is deployed within vehicles 218, sensor 206 may collect event data relating to any treatment or healthcare services rendered to patient 204 while in vehicles 218.

Medical care facility 202 may also include identification tag 220. Identification tag 220 is one or more tags associated with objects or persons in medical care facility 202. For example, identification tag 220 may be utilized to identify an object or person and to determine a location of the object or person. For example, identification tag 220 may be, without limitation, a bar code pattern, such as a universal product code (UPC) or European article number (EAN), a radio frequency identification (RFID) tag, or other optical identification tag. The type of identification tag implemented in medical care facility 202 depends upon the capabilities of the image capture device and associated data processing system to process the information.

Sensor 206 may be any type of sensing device for gathering event data associated with the delivery of healthcare services at patient care environment 200. Sensor 206 may include, without limitation, a camera, a motion sensor device, a sonar, a sound recording device, an audio detection device, a voice recognition system, a heat sensor, a seismograph, a pressure sensor, a device for detecting odors, scents, and/or fragrances, a radio frequency identification (RFID) tag reader, a global positioning system (GPS) receiver, and/or any other detection device for detecting the presence of a human, animal, equipment, or vehicle at patient care environment 200.

A heat sensor may be any type of known or available sensor for detecting body heat generated by a human or animal. A heat sensor may also be a sensor for detecting heat generated by a vehicle, such as an automobile or a motorcycle.

A motion detector may include any type of known or available motion detector device. A motion detector device may include, but is not limited to, a motion detector device using a photo-sensor, radar or microwave radio detector, or ultrasonic sound waves.

A motion detector using ultrasonic sound waves transmits or emits ultrasonic sounds waves. The motion detector detects or measures the ultrasonic sound waves that are reflected back to the motion detector. If a human, animal, or other object moves within the range of the ultrasonic sound waves generated by the motion detector, the motion detector detects a change in the echo of sound waves reflected back. This change in the echo indicates the presence of a human, animal, or other object moving within the range of the motion detector.

In one example, a motion detector device using a radar or microwave radio detector may detect motion by sending out a burst of microwave radio energy and detecting the same microwave radio waves when the radio waves are deflected back to the motion detector. If a human, animal, or other object moves into the range of the microwave radio energy field generated by the motion detector, the amount of energy reflected back to the motion detector is changed. The motion detector identifies this change in reflected energy as an indication of the presence of a human, animal, or other object moving within the motion detectors range.

A motion detector device, using a photo-sensor, detects motion by sending a beam of light across a space into a photo-sensor. The photo-sensor detects when a human, animal, or object breaks or interrupts the beam of light as the human, animal, or object moves in-between the source of the beam of light and the photo-sensor. These examples of motion detectors are presented for illustrative purposes only. A motion detector in accordance with the illustrative embodiments may include any type of known or available motion detector and is not limited to the motion detectors described herein.

A pressure sensor detector may be, for example, a device for detecting a change in weight or mass associated with the pressure sensor. For example, if one or more pressure sensors are imbedded in a sidewalk, Astroturf, or floor mat, the pressure sensor detects a change in weight or mass when a human or animal steps on the pressure sensor. The pressure sensor may also detect when a human or animal steps off of the pressure sensor. In another example, one or more pressure sensors are embedded in a parking lot, and the pressure sensors detect a weight and/or mass associated with a vehicle when the vehicle is in contact with the pressure sensor. A vehicle may be in contact with one or more pressure sensors when the vehicle is driving over one or more pressure sensors and/or when a vehicle is parked on top of one or more pressure sensors.

A camera may be any type of known or available camera, including, but not limited to, a video camera for taking moving video images, a digital camera capable of taking still pictures and/or a continuous video stream, a stereo camera, a web camera, and/or any other imaging device capable of capturing a view of whatever appears within the camera's range for remote monitoring, viewing, or recording of a distant or obscured person, object, or area.

Various lenses, filters, and other optical devices such as zoom lenses, wide angle lenses, mirrors, prisms and the like may also be used with the image capture device to assist in capturing the desired view. Devices may be fixed in a particular orientation and configuration, or it may, along with any optical device, be programmable in orientation, light sensitivity level, focus or other parameters. Programming data may be provided via a computing device, such as server 104 in FIG. 1.

A camera may also be a stationary camera and/or a non-stationary camera. A non-stationary camera is a camera that is capable of moving and/or rotating along one or more directions, such as up, down, left, right, and/or rotate about an axis of rotation. The camera may also be capable of moving to follow or track a person, animal, or object in motion. In other words, the camera may be capable of moving about an axis of rotation in order to keep a patient, healthcare professional, animal, or object within a viewing range of the camera lens. In this example, sensor 206 includes non-stationary digital video cameras.

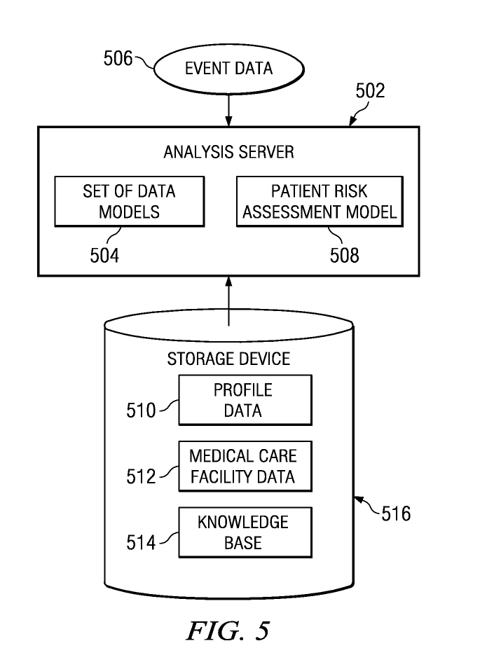

Sensor 206 is coupled to, or in communication with, an analysis server on a data processing system, such as network data processing system 100 in FIG. 1. The analysis server is illustrated and described in greater detail in FIG. 5, below. The analysis server includes software for analyzing digital images and other data captured by sensor 206 to gather event data in patient care environment 200.

The data collected by sensor 206 is sent to smart detection software. The smart detection software processes the data to form the event data. The event data includes data and metadata describing events captured by sensor 206. The event data may be combined with static data and sent to the analysis server for additional processing to identify events affecting a patient that occur in patient care environment 200. Once events affecting the patient are identified, the events may be parsed to form assessment data usable to generate a patient risk assessment model.

Assessment data is data relevant to an assessment of a patient. An assessment of a patient is the identification of a cause of some condition of the patient. Thus, the assessment of a patient may be, for example, an identification of a disease from the symptoms manifested in the patient to cause the patient's condition. In addition, an assessment of a patient may also be identification of a prior event or action causing a condition of a patient. For example, a patient may have fallen into a coma because of an adverse reaction to medication previously administered to the patient. The coma is the condition of the patient and the administered medication is the prior event causing the condition.

Similarly, events or conditions that are not relevant to an assessment of a patient may be event data, but are not assessment data. For example, the patient who had fallen into a coma because of an adverse reaction to medication may have received a sponge bath prior to the administration of the medication. The sponge bath, however, in no way contributed to the patient's coma. Thus, the sponge bath is not relevant to the assessment of the patient and is therefore not assessment data.

The data processing system, discussed in greater detail in FIG. 3 below, includes associated memory, which may be an integral part, such as the operating memory, of the data processing system or externally accessible memory. Software for tracking objects may reside in the memory and run on the processor. The software in the data processing system keeps a list of all patients, personnel, medications, sensors, equipment, and any other person or item of interest in medical care facility 202. The list is stored in a database. The database may be any type of database such as a spreadsheet, relational database, hierarchical database or the like. The database may be stored in the operating memory of the data processing system, externally on a secondary data storage device, locally on a recordable medium such as a hard drive, floppy drive, CD ROM, DVD device, remotely on a storage area network, such as storage 108 in FIG. 1, or in any other type of storage device.

The lists are updated frequently enough to maintain a dynamic, accurate, real time listing of the people and objects within medical care facility 202 and patient care environment 200. Further, the lists maintain a real time listing of the events occurring within medical care facility 202. The listing of people, objects, and events may be used to trigger predefined actions. For example, a patient monitoring system may generate an alert if the patient monitoring system detects that a medical care worker is attempting to administer the wrong medication to a patient. In another example, the patient monitoring system may generate an alert for receipt by a medical care worker alerting the medical care worker that a patient had not yet received a required medication or a meal.

With reference now to FIG. 3, a block diagram of a data processing system is shown in which illustrative embodiments may be implemented. Data processing system 300 is an example of a computer, such as server 104 and client 110 in FIG. 1, in which computer usable program code or instructions implementing the processes may be located for the illustrative embodiments.

In the depicted example, data processing system 300 employs a hub architecture including a north bridge and memory controller hub (NB/MCH) 302 and a south bridge and input/output (I/O) controller hub (SB/ICH) 304. Processing unit 306, main memory 308, and graphics processor 310 are coupled to north bridge and memory controller hub 302. Processing unit 306 may contain one or more processors and may even be implemented using one or more heterogeneous processor systems. Graphics processor 310 may be coupled to NB/MCH 302 through an accelerated graphics port (AGP), for example.

In the depicted example, local area network (LAN) adapter 312 is coupled to south bridge and I/O controller hub 304 and audio adapter 316, keyboard and mouse adapter 320, modem 322, read only memory (ROM) 324, universal serial bus (USB) and other ports 332, and PCI/PCIe devices 334 are coupled to south bridge and I/O controller hub 304 through bus 338, and hard disk drive (HDD) 326 and CD-ROM 330 are coupled to south bridge and I/O controller hub 304 through bus 340. PCI/PCIe devices may include, for example, Ethernet adapters, add-in cards, and PC cards for notebook computers. PCI uses a card bus controller, while PCIe does not. ROM 324 may be, for example, a flash binary input/output system (BIOS). Hard disk drive 326 and CD-ROM 330 may use, for example, an integrated drive electronics (IDE) or serial advanced technology attachment (SATA) interface. A super I/O (SIO) device 336 may be coupled to south bridge and I/O controller hub 304.

An operating system runs on processing unit 306 and coordinates and provides control of various components within data processing system 300 in FIG. 3. The operating system may be a commercially available operating system such as Microsoft® Windows® XP (Microsoft and Windows are trademarks of Microsoft Corporation in the United States, other countries, or both). An object oriented programming system, such as the JAVA™ programming system, may run in conjunction with the operating system and provides calls to the operating system from JAVA™ programs or applications executing on data processing system 300. JAVA™ and all JAVA™-based trademarks are trademarks of Sun Microsystems, Inc. in the United States, other countries, or both.

Instructions for the operating system, the object-oriented programming system, and applications or programs are located on storage devices, such as hard disk drive 326, and may be loaded into main memory 308 for execution by processing unit 306. The processes of the illustrative embodiments may be performed by processing unit 306 using computer implemented instructions, which may be located in a memory such as, for example, main memory 308, read only memory 324, or in one or more peripheral devices.

In some illustrative examples, data processing system 300 may be a personal digital assistant (PDA), which is generally configured with flash memory to provide non-volatile memory for storing operating system files and/or user-generated data. A bus system may be comprised of one or more buses, such as a system bus, an I/O bus and a PCI bus. Of course the bus system may be implemented using any type of communications fabric or architecture that provides for a transfer of data between different components or devices attached to the fabric or architecture. A communications unit may include one or more devices used to transmit and receive data, such as a modem or a network adapter. Memory may be, for example, main memory 308 or a cache such as found in north bridge and memory controller hub 302. A processing unit may include one or more processors or CPUs. The depicted examples in FIGS. 1 and 3 and in the above-described examples are not meant to imply architectural limitations. For example, data processing system 300 may also be a tablet computer, laptop computer, or telephone device in addition to taking the form of a PDA.

A director, operator, manager or other employee associated with patient care environment 114 in FIG. 1 typically has a need to identify causes of morbidity and mortality in a medical care facility. Once identified, preventable causes of morbidity and mortality may be eliminated or reduced. In addition, identification of causes of morbidity and mortality may also allow medical care workers to effectively treat patients. Therefore, the aspects of the illustrative embodiments recognize that it is advantageous for a director or other employee of the medical care environment to have a patient risk assessment model that takes into account as much information regarding patients, medical care workers, and events occurring in a medical care facility to assist in the provision of healthcare services to patients, and to facilitate the assessment and treatment of patients.

Therefore, the illustrative embodiments described herein provide a computer implemented method, apparatus, and computer usable program product for generating a risk assessment model for a patient in a healthcare facility. The process retrieves event data for the patient, wherein the event data is derived from video data, and wherein the event data further comprises metadata describing events affecting the patient in a medical care facility, and parses the event data to form assessment data. The process then generates the risk assessment model using the assessment data.

It will be appreciated by one skilled in the art that the words “optimize”, “optimization”, and related terms are terms of art that refer to improvements in speed and/or efficiency of a computer program, and do not purport to indicate that a computer program has achieved, or is capable of achieving, an “optimal” or perfectly speedy/perfectly efficient state.

A patient risk assessment model is a model that identifies a set of patient morbidity factors. The set of patient morbidity factors is one or more factors that may be attributed to the morbidity or mortality of a patient in a medical care facility. The factors may be events, actions, omissions, or conditions that cause the morbidity or mortality of a patient. For example, regarding a patient who died from an accidental overdose of medication, the patient morbidity factor may include an identification of the medication, the amount of medication administered, the identity of the medical care worker that administered the medication, and the actual affect the medication had on the patient. For example, the overdose may have stopped the patient's heart, in which case the set of patient morbidity factors may also indicate that the patient suffered from an irrecoverable heart condition.

In addition, the patient risk assessment model may suggest one or more remedies to address the set of patient morbidity factors. For example, the patient risk assessment model for a patient that has fallen ill after surgery may indicate that the cause of the illness was improperly sterilized equipment. Thus, the patient risk assessment model may also provide guidelines for proper sterilization of equipment. In other words, the patient risk assessment model may also provide information, suggestions, and instructions for one or more patient treatment strategies. In addition, the patient risk assessment model may also provide a remedy to treat the patient's illness that resulted from the improperly sterilized equipment.

A patient treatment strategy is a set of one or more actions usable by a medical care worker to treat a patient's condition or the cause of the patient's condition. For example, where a risk assessment model of a patient identifies the cause of a patient's allergic reaction, a patient treatment strategy may be developed that includes isolation from the source of the allergen and the provision of one or more anti-allergy medications.

Processing or parsing event data to generate the patient risk assessment model may include, but is not limited to, formatting the event data for utilization and/or analysis in one or more data models, combining the event data with secondary sources of data, comparing the event data to a data model and/or filtering the event data for relevant data elements to form the dynamic data. Secondary sources of data may include, for example, medical care facility databases storing patient records, staffing records, billing records, and test results. Secondary data sources may also be knowledge bases that include, for example, publicly available information or information released by a third party, such as a drug or equipment manufacturer.

As used herein, the term “set” includes one or more. For example, a set of motion detectors may include a single motion detector or two or more motion detectors. In one embodiment, the detectors include a set of one or more cameras located externally to the medical care facility. Video images received from the set of cameras are used for gathering event data used to create the patient risk assessment model for a patient at the medical care facility.

Event data collected from a set of sensors in a detection system is used to generate the patient risk assessment model. The set of sensors, which may be one or more sensors, is configured to monitor an environment. The environment may be a medical care facility, such as a hospital, nursing home, or personal residence.

Dynamic data is data relating to patients or healthcare workers that is gathered and analyzed in real time as healthcare services are rendered. Dynamic data is data that has been processed or filtered for analysis in a data model. For example, a data model may not be capable of analyzing raw, or unprocessed video images captured by a camera. The video images may need to be processed into data and/or metadata describing the contents of the video images before a data model may be used to organize, structure, or otherwise manipulate data and/or metadata. The video images converted to data and/or metadata that are ready for processing or analysis in a set of data models is an example of dynamic data.

The dynamic data is analyzed using one or more data models in a set of data models to identify events affecting a patient in the medical care facility. The events may include, for example, treatment provided to a patient, the identity of the medical care worker who administered the treatment, an amount of time that elapsed since medications were provided, the activities performed by a patient or healthcare worker, symptoms exhibited by a patient, and the events that may have caused the onset of the symptoms.

Dynamic data describes events that directly or indirectly affect a patient. Events that directly affect a patient are those actions or events that are directed specifically to a patient. For example, events that directly affect a patient may include the delivery of a meal to a patient, the evaluation of a patient by a doctor, the administration of treatments to a patient, the manifestation of a condition or symptom by a patient, or any other event or action that directly involves a patient.

Dynamic data also includes events that indirectly affect a patient. Events that indirectly affect a patient are those events or actions that affect a patient, but which were not directed specifically toward the patient. Thus, for example, an event that indirectly affects a patient may be the contamination of a water supply that resulted in the patient contracting a bacterial infection. Other events that indirectly affect a patient may include the accidental destruction of hospital gowns that prevents the patient from receiving proper hospital attire, or the mislabeling of a drug that was then administered to the patient.

The events may be further processed in one or more data models in the set of data models to generate the patient risk assessment model. The patient risk assessment model may include a set of patient morbidity factors and a set of treatment options. The set of treatment options are one or more treatments provided by the risk assessment model to address the set of patient morbidity factors. The set of patient morbidity factors and treatments are usable by a doctor or other medical care professional to identify causes of morbidity and mortality and to formulate a treatment strategy to assist the patient.

A set of data models includes one or more data models. A data model is a model for structuring, defining, organizing, imposing limitations or constraints, and/or otherwise manipulating data and metadata to produce a result. A data model may be generated using any type of modeling method or simulation including, but not limited to, a statistical method, a data mining method, a causal model, a mathematical model, a behavioral model, a psychological model, a sociological model, or a simulation model.

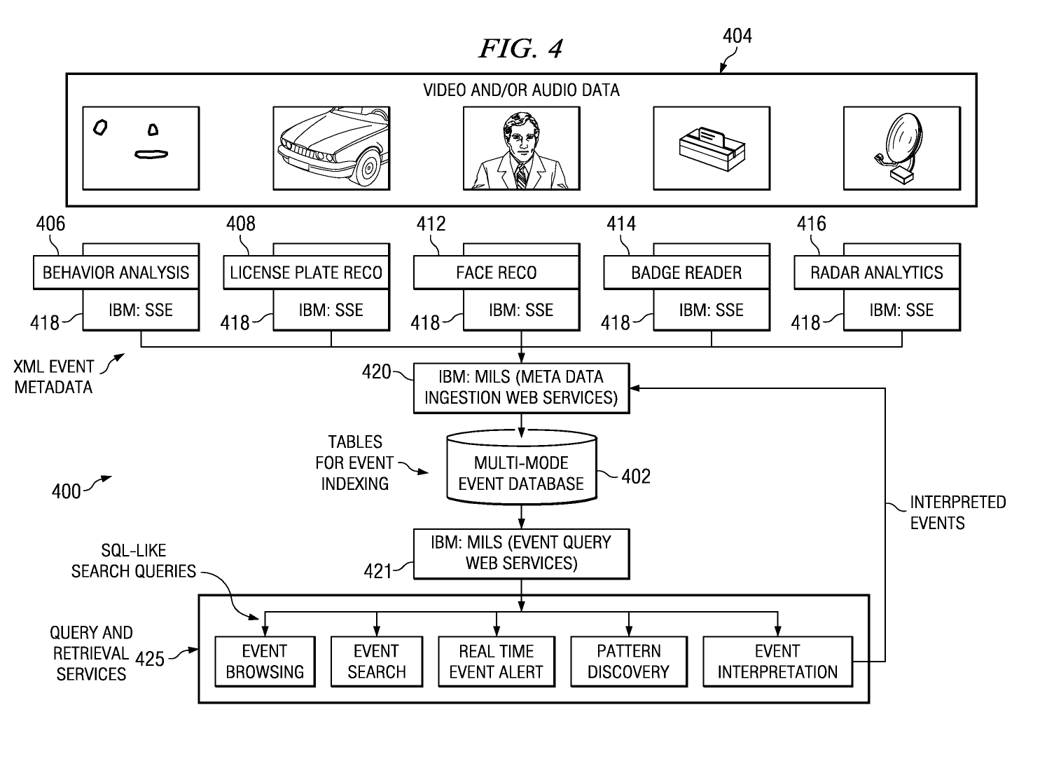

Turning now to FIG. 4, a diagram of a smart detection system is depicted in accordance with an illustrative embodiment. System 400 is a system, such as network data processing system 100 in FIG. 1. System 400 incorporates multiple independently developed event analysis technologies in a common framework. An event analysis technology is a collection of hardware and/or software usable to capture and analyze event data. For example, an event analysis technology may be the combination of a video camera and facial recognition software. Images of faces captured by the video camera are analyzed by the facial recognition software to identify the subjects of the images.

Smart detection, also known as smart surveillance, is the use of computer vision and pattern recognition technologies to analyze detection data gathered from situated cameras and microphones. The analysis of the detection data generates events of interest in the environment. For example, an event of interest at a departure drop off area in an airport includes “cars that stop in the loading zone for extended periods of time.” As smart detection technologies have matured, they have typically been deployed as isolated applications which provide a particular set of functionalities.

Smart detection system 400 is a smart detection system architecture for analyzing video images captured by a camera and/or audio captured by an audio detection device. Smart detection system 400 includes software for analyzing audio/video data 404. In this example, smart detection system 400 processes audio/video data 404 for a patient or healthcare professional into data and metadata to form query and retrieval services 425. Smart detection system 400 may be implemented using any known or available software for performing voice analysis, facial recognition, license plate recognition, and sound analysis. In this example, smart detection system 400 is implemented as IBM® smart surveillance system (S3) software.

An audio/video capture device is any type of known or available device for capturing video images and/or capturing audio. The audio/video capture device may be, but is not limited to, a digital video camera, a microphone, a web camera, or any other device for capturing sound and/or video images. For example, the audio/video capture device may be implemented as sensor 206 in FIG. 2.

Audio/video data 404 is detection data captured by the audio/video capture devices. Audio/video data 404 may be a sound file, a media file, a moving video file, a media file, a still picture, a set of still pictures, or any other form of image data and/or audio data. Audio/video data 404 may also be referred to as detection data. Audio/video data 404 may include images of a person's face, an image of a part or portion of a car, an image of a license plate on a car, and/or one or more images showing a person's behavior. For example, a set of images showing a patient's behavior or appearance may indicate that a patient is responding poorly to a particular form of treatment or type of medication.

In this example, smart detection system 400 architecture is adapted to satisfy two principles. 1) Openness: The system permits integration of both analysis and retrieval software made by third parties. In one embodiment, the system is designed using approved standards and commercial off-the-shelf (COTS) components. 2) Extensibility: The system should have internal structures and interfaces that will permit the functionality of the system to be extended over a period of time.

The architecture enables the use of multiple independently developed event analysis technologies in a common framework. The events from all these technologies are cross indexed into a common repository or multi-mode event database multi-mode event 402 allowing for correlation across multiple audio/video capture devices and event types.

Smart detection system 400 includes the following illustrative technologies integrated into a single system. License plate recognition technology 408 may be deployed at the entrance to a facility where license plate technology 408 catalogs a license plate of each of the arriving and departing vehicles in a parking lot associated with the medical care facility.

Behavior analysis technology 406 detects and tracks moving objects and classifies the objects into a number of predefined categories. As used herein, an object may be a human patient or healthcare professional, an item, such as medical equipment or tools, or any other item located inside or outside the medical care facility. Behavior analysis technology 406 could be deployed on various cameras overlooking a parking lot, a perimeter, or inside a facility.

Face detection/recognition technology 412 may be deployed at entry ways to capture and recognize faces. Badge reading technology 414 may be employed to read badges. Radar analytics technology 416 may be employed to determine the presence of objects.

Events from access control technologies can also be integrated into smart detection system 400. The data gathered from behavior analysis technology 406, license plate recognition 408, face detection/recognition technology 412, badge reader technology 414, radar analytics technology 416, and any other video/audio data received from a camera or other video/audio capture device is received by smart detection system 400 for processing into query and retrieval services 425.

The events from all the above surveillance technologies are cross indexed into a single repository, such as multi-mode event database 402. In such a repository, a simple time range query across the modalities will extract license plate information, vehicle appearance information, badge information and face appearance information, thus permitting an analyst to easily correlate these attributes. The architecture of smart detection system 400 also includes one or more smart surveillance engines (SSEs) 418, which house event detection technologies.

Smart detection system 400 further includes Middleware for Large Scale Surveillance (MILS) 420 and 421, which provides infrastructure for indexing, retrieving and managing event metadata.

In this example, audio/video data 404 is received from a variety of audio/video capture devices, such as sensor 206, and processed in SSEs 418. Each SSE 418 can generate real time alerts and generic event metadata. The metadata generated by SSE 418 may be represented using extensible markup language (XML). The XML documents include a set of fields which are common to all engines and others which are specific to the particular type of analysis being performed by SSE 418. In this example, the metadata generated by SSEs 418 is transferred to a backend MILS system 420. This may be accomplished via the use of, e.g., web services data ingest application program interfaces (APIs) provided by MILS 420. The XML metadata is received by MILS 420 and indexed into predefined tables in database multi-mode event 402. This may be accomplished using, for example, and without limitation, the DB2™ XML extender, if an IBM® DB2™ database is employed. This permits for fast searching using primary keys. MILS 421 provide query and retrieval services 425 based on the types of metadata available in the database. Query and retrieval services 425 may include, for example, event browsing, event search, real time event alert, or pattern discovery event interpretation. Each event has a reference to the original media resource, such as, without limitation, a link to the video file. This allows a user to view the video associated with a retrieved event.

Smart detection system 400 provides an open and extensible architecture for smart video surveillance. SSEs 418 preferably provide a plug and play framework for video analytics. The event metadata generated by SSEs 418 may be sent to multi-mode event database 402 as XML files. Web services API's in MILS 420 permit for easy integration and extensibility of the metadata. Query and retrieval services 425, such as, for example, event browsing and real time alerts, may use structure query language (SQL) or similar query language through web services interfaces to access the event metadata from multi-mode event database 402.

The smart surveillance engine (SSE) 418 may be implemented as a C++ based framework for performing real time event analysis. SSE 418 is capable of supporting a variety of video/image analysis technologies and other types of sensor analysis technologies. SSE 418 provides at least the following support functionalities for the core analysis components. The support functionalities are provided to programmers or users through a plurality of interfaces employed by the SSE 418. These interfaces are illustratively described below.

Standard plug-in interfaces are provided. Any event analysis component which complies with the interfaces defined by SSE 418 can be plugged into SSE 418. The definitions include standard ways of passing data into the analysis components and standard ways of getting the results from the analysis components. Extensible metadata interfaces are provided. SSE 418 provides metadata extensibility. For example, consider a behavior analysis application which uses detection and tracking technology. Assume that the default metadata generated by this component is object trajectory and size. If the designer now wishes to add color of the object into the metadata, SSE 418 enables this by providing a way to extend the creation of the appropriate XML structures for transmission to the backend (MILS) system 420.

Real time alerts are highly application-dependent. For example, while a person loitering may require an alert in one application, the absence of a guard at a specified location may require an alert in a different application. The SSE provides an easy real time alert interface mechanism for developers to plug-in for application specific alerts. SSE 418 provides standard ways of accessing event metadata in memory and standardized ways of generating and transmitting alerts to the backend (MILS) system 420.

In many applications, users will need the use of multiple basic real time alerts in a spatio-temporal sequence to compose an event that is relevant in the user's application context. SSE 418 provides a simple mechanism for composing compound alerts via compound alert interfaces. In many applications, the real time event metadata and alerts are used to actuate alarms, visualize positions of objects on an integrated display and control cameras to get better surveillance data. SSE 418 provides developers with an easy way to plug-in actuation modules which can be driven from both the basic event metadata and by user defined alerts using real time actuation interfaces.

Using database communication interfaces, SSE 418 also hides the complexity of transmitting information from the analysis engines to the multi-mode event database 402 by providing simple calls to initiate the transfer of information.

The IBM middleware for large scale surveillance (MILS) 420 and 421 may include a J2EE™ frame work built around IBM's DB2™ and IBM WebSphere™ application server platforms. MILS 420 supports the indexing and retrieval of spatio-temporal event metadata. MILS 420 also provides analysis engines with the following support functionalities via standard web service interfaces using XML documents.

MILS 420 and 421 provide metadata ingestion services. These are web service calls which allow an engine to ingest events into the MILS 420 and 421 system. There are two categories of ingestion services. 1) Index Ingestion Services This permits for the ingestion of metadata that is searchable through SQL like queries. The metadata ingested through this service is indexed into tables which permit content based searches, such as provided by MILS 420. 2) Event Ingestion Services: This permits for the ingestion of events detected in SSE 418, such as provided by MILS 421. For example, a loitering alert that is detected can be transmitted to the backend along with several parameters of the alert. These events can also be retrieved by the user but only by the limited set of attributes provided by the event parameters.

The MILS 420 and/or 421 provides schema management services. Schema management services are web services which permit a developer to manage their own metadata schema. A developer can create a new schema or extend the base MILS schema to accommodate the metadata produced by their analytical engine. In addition, system management services are provided by the MILS 420 and/or 421.

The schema management services of MILS 420 and 421 provide the ability to add a new type of analytics to enhance situation awareness through cross correlation. For example, a patient risk assessment model associated with a patient is dynamic and can change over time. For example, treatment strategies may vary by season, or may change drastically because of the advent of new medical equipment, medications, or procedures. Thus, it is important to permit smart detection system 400 to add new types of analytics and cross correlate the existing analytics with the new analytics. To add/register a new type of sensor and/or analytics to increase situation awareness, a developer can develop new analytics and plug them into SSE 418, and employ MILS's schema management service to register new intelligent tags generated by the new SSE analytics. After the registration process, the data generated by the new analytics is immediately available for cross correlating with existing index data.

System management services provide a number of facilities needed to manage smart detection system 400 including: 1) Camera Management Services: These services include the functions of adding or deleting a camera from a MILS system, adding or deleting a map from a MILS system, associating a camera with a specific location on a map, adding or deleting views associated with a camera, assigning a camera to a specific MILS server and a variety of other functionalities needed to manage the system. 2) Engine Management Services: These services include functions for starting and stopping an engine associated with a camera, configuring an engine associated with a camera, setting alerts on an engine and other associated functionalities. 3) User Management Services: These services include adding and deleting users with a system, associating selected cameras with a viewer, associating selected search and event viewing capacities with a user and associating video viewing privilege with a user. 4) Content Based Search Services: These services permit a user to search through an event archive using a plurality of types of queries.

For the content based search services (4), the types of queries may include: A) Search by Time retrieves all events from query and retrieval services 425 that occurred during a specified time interval. B) Search by Object Presence retrieves the last 100 events from a live system. C) Search by Object Size retrieves events where the maximum object size matches the specified range. D) Search by Object Type retrieves all objects of a specified type. E) Search by Object Speed retrieves all objects moving within a specified velocity range. F) Search by Object Color retrieves all objects within a specified color range. G) Search by Object Location retrieves all objects within a specified bounding box in a camera view. H) Search by Activity Duration retrieves all events from query and retrieval services 425 with durations within the specified range. I) Composite Search combines one or more of the above capabilities. Other system management services may also be employed.

Referring now to FIG. 5, a block diagram of a data processing system for analyzing event data for event patterns utilized to generate a healthcare delivery model is shown in accordance with an illustrative embodiment. Data processing system 500 is a data processing system, such as data processing system 100 in FIG. 1 and data processing system 300 in FIG. 3.

Analysis server 502 is any type of known or available server for analyzing data for use in generating a patient risk assessment model. Analysis server 502 may be a server, such as server 104 in FIG. 1 or data processing system 300 in FIG. 3. Analysis server 502 includes a set of data models 504 for analyzing dynamic data elements and static data elements.

Static data elements are data elements that do not tend to change in real time. Examples of static data elements include, without limitation, a patient's name, a patient's medical history, a healthcare worker's name, a healthcare worker's certifications, and a payroll. For example, static data elements may be collected from administrative records and paperwork, for example. Static data elements may be stored in hospital databases, public servers, knowledgebases, or any other location.

Dynamic data elements are data elements that are changing in real time. For example, dynamic data elements could include, without limitation, the identity of patients and personnel located at a medical care facility, actions performed by patients and healthcare workers, medications and treatments provided to patients, the time of day, the day of the week, the temperature of the medical care facility, lighting conditions, the level of contamination in a room, and the movement of people throughout the medical care facility. Event data is a dynamic data element. Additionally, dynamic data elements may be collected by sensors deployed at a medical care facility, such as sensor 206 in FIG. 2. Dynamic data elements and static data elements may be combined to form dynamic data.

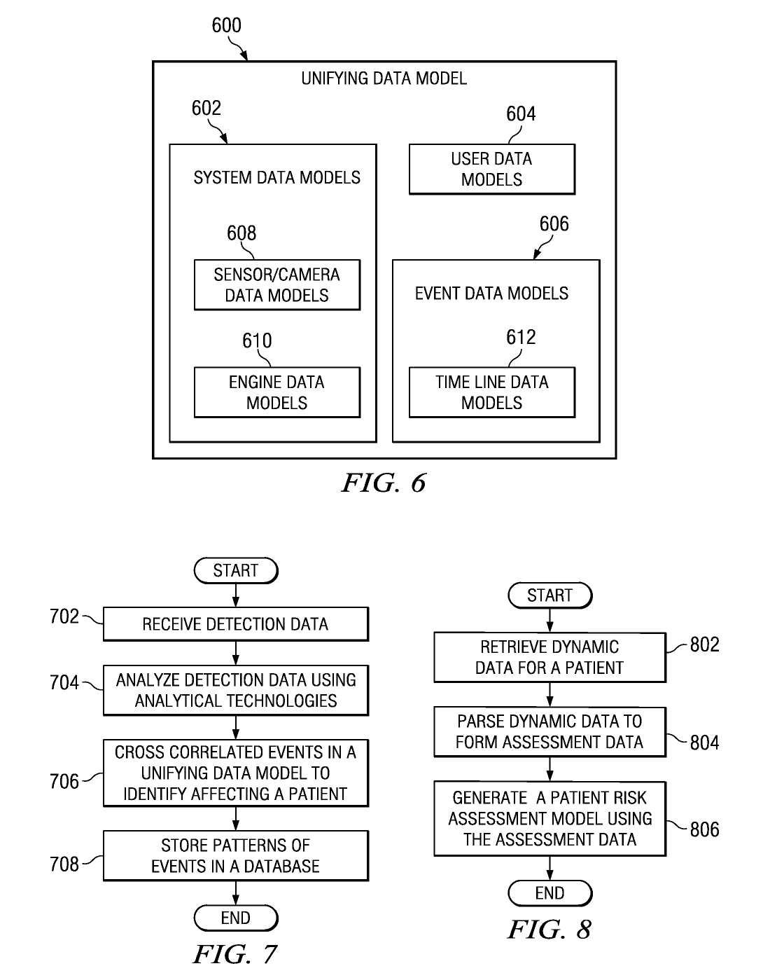

Set of data models 504 is one or more data models created a priori or pre-generated for use in analyzing event data 506 to identify event patterns and generate patient risk assessment model 508. Set of data models 504 includes one or more data models for mining event data, identifying events of interest, and determining patterns or relationships between the events of interest. Set of data models 504 are generated using statistical, data mining, and simulation or modeling techniques. In this example, set of data models 504 includes, but is not limited to, a unifying data model, system data models, event data models, and/or user data models. These data models are discussed in greater detail in FIG. 6 below.

Event data 506 is a model, set of definitions, suggestions, or parameters for use in implementing a patient treatment strategy in a medical care environment. Event data 506 may identify a set of patient morbidity factors and suggested treatments for remedying the patient morbidity factors, as described above.

Profile data 510 is data relating to one or more persons that may be found in a medical care facility. For example, profile data 510 may relate to a healthcare worker, a patient, or even family and friends of a patient. For a patient, profile data 510 may include patient medical records, patient preferences, family histories, and any records, patient preferences, family histories, and any other information that would be relevant in the patient's risk assessment. For a healthcare worker, profile data 510 may include a preferred work schedule, known physical limitations that may prevent the performance of certain tasks, lists of certifications obtained, and previous job descriptions.

Event data 506 is data or metadata describing events occurring in the patient care environment. Event data 506 is processed to form dynamic data. Dynamic data includes events that occur in a patient care environment. Processing event data 506 may include, but is not limited to, parsing event data 506 for relevant data elements, combining event data 506 with data profile data 510, medical care facility data 512, or knowledge base 514. In addition, processing event data 506 may include comparing event data 506 to baseline or comparison models, and/or formatting event data 506 for utilization and/or analysis in one or more data models in a set of data models 504 to form the dynamic data. The processed event data 506 and any other data forms dynamic data (not shown). The dynamic data, which includes events of interest, is analyzed and/or further processed using one or more data models in set of data models 504 to generate patient risk assessment model 508.

Medical care facility data 512 is data generated by, maintained at, or otherwise associated with the medical care facility from which event data 506 may be generated. Medical care facility data 512 includes, for example, staffing records, billing records, inventory databases, and any other type of data that may be relevant to the generation of patient risk assessment model 508.

Knowledge base 514 is data that is not directly associated with the medical care facility, but which may be relevant in the generation of patient risk assessment model 508. For example, knowledge base 514 may contain publicly available information, such as known drug interactions and side effects, optimal medication dosages for treating specific illnesses, material safety data sheets, or any other form of publicly available information that may be relevant to a patient's risk assessment and treatment. Additionally, knowledge base 514 may also include, for example, confidential data available from third parties, such as research facilities and drug manufacturers.

Profile data 510, medical care facility data 512, and knowledge base 514 are stored in storage device 516. Storage device 516 is one or more storage devices for storing data, such as storage 108 in FIG. 1 and hard disk drive 326 in FIG. 3. In another embodiment, profile data 510, medical care facility data 512, and knowledge base 514 may be stored in separate storage devices of the same computer system, or may be stored in storage devices located on a network.

Turning now to FIG. 6, a block diagram of a unifying data model for processing event data is depicted in accordance with an illustrative embodiment. The event data generated by a smart detection system may be processed by one or more data models in a set of data models, such as set of data models 504 in FIG. 5, to identify patterns in the events. Unifying data model 600 is an example of a data model for processing event data.

In this example, unifying data model 600 has three types of data models, namely, 1) system data models 602 which captures the specification of a given monitoring system, including details like geographic location of the system, number of cameras deployed in the system, physical layout of the monitored space, and other details regarding the patient care environment and medical care facility; 2) user data models 604 models users, privileges and user functionality; and 3) event data models 606 which captures the events that occur in a specific sensor or zone in the monitored space. Each of these data models is described below.

System data models 602 has a number of components. These may include sensor/camera data models 608. The most fundamental component of models 608 is a view. A view is defined as some particular placement and configuration, such as a location, orientation, and/or parameters, of a sensor. In the case of a camera, a view would include the values of the pan, tilt and zoom parameters, any lens and camera settings and position of the camera. A fixed camera can have multiple views. The view “Id” may be used as a primary key to distinguish between events being generated by different sensors. A single sensor can have multiple views. Sensors in the same geographical vicinity are grouped into clusters, which are further grouped under a root cluster. There is one root cluster per MILS server.

Engine data models 610 provide a comprehensive security solution which utilizes a wide range of event detection technologies. Engine data model 610 captures at least some of the following information about the analytical engines: Engine Identifier: A unique identifier assigned to each engine; Engine Type: This denotes the type of analytic being performed by the engine, for example face detection, behavior analysis, and/or LPR; and Engine Configuration: This captures the configuration parameters for a particular engine.

User data models 604 captures the privileges of a given user. These may include selective access to camera views; selective access to camera/engine configuration and system management functionality; and selective access to search and query functions.

Event data models 606 represents the events that occur within a space that may be monitored by one or more cameras or other sensors. Event data model may incorporate time line data models 612 for associating the events with a time. By associating the events with a time, an integrated event may be defined. An integrated event is an event that may include multiple sub-events. Time line data model 612 uses time as a primary synchronization mechanism for events that occur in the real world, which is monitored through sensors. The basic MILS schema allows multiple layers of annotations for a given time span.

Turning now to FIG. 7, a process for generating event data by a smart detection system is depicted in accordance with an illustrative embodiment. The process in FIG. 7 may be implemented by a smart detection system, such as smart detection system 400 in FIG. 4.

The process begins by receiving detection data from a set of cameras (step 702). The process analyzes the detection data using multiple analytical technologies to detect events (step 704). The multiple technologies may include, for example, a behavior analysis engine, a license plate recognition engine, a face recognition engine, a badge reader engine, and/or a radar analytic engine.

Events are cross correlated in a unifying data model to identify events affecting a patient (step 706). Cross correlating provides integrated situation awareness across the multiple analytical technologies. The cross correlating may include correlating events to a time line to associate events to define an integrated event. The events are indexed and stored in a repository, such as a database (step 708) with the process terminating thereafter.

In the example in FIG. 7, the database can be queried to determine an integrated event that matches the query. This includes employing cross correlated information from a plurality of information technologies and/or sources. New analytical technologies may also be registered. The new analytical technologies can employ models and cross correlate with existing analytical technologies to provide a dynamically configurable surveillance system.

In this example, detection data is received from a set of cameras. However, in other embodiments, detection data may come from other detection devices, such as, without limitation, a badge reader, a microphone, a motion detector, a heat sensor, or a radar.

Turning now to FIG. 8, a process for generating a patient risk assessment model is depicted in accordance with an illustrative embodiment. This process may be implemented by an analysis server, such as analysis server 502 in FIG. 5.

The process begins by retrieving dynamic data for a patient (step 802). The dynamic data may be retrieved from a data storage device, such as a relational database, a multimode database, or any other data storage device. The dynamic data includes metadata describing events affecting a patient at a medical care facility. The dynamic data may include event data collected from a set of sensors deployed at a medical care facility usable to monitor a patient. The dynamic data may also include static data elements collected from a public database or a database maintained by a medical care facility, or a third party entity.

The process then parses the dynamic data to form assessment data (step 804). The process then generates a risk assessment model using the assessment data (step 806). The risk assessment model may be used to formulate a patient treatment strategy. The patient treatment strategy may provide a suggestion to remedy a condition of a patient, or may provide suggestions as to how to take preventative measures to avoid the occurrence of the condition in the future.

The flowcharts and block diagrams in the different depicted embodiments illustrate the architecture, functionality, and operation of some possible implementations of methods, apparatus, and computer program products. In this regard, each block in the flowchart or block diagrams may represent a module, segment, or portion of code, which comprises one or more executable instructions for implementing the specified function or functions. In some alternative implementations, the function or functions noted in the block may occur out of the order noted in the figures. For example, in some cases, two blocks shown in succession may be executed substantially concurrently, or the blocks may sometimes be executed in the reverse order, depending upon the functionality involved.

The illustrative embodiments described herein provide a computer implemented method, apparatus, and computer program product for generating a patient risk assessment model for a patient at a medical care facility. The process retrieves event data for the patient, wherein the event data is derived from video data, and wherein the event data further comprises metadata describing events affecting the patient in a medical care facility, and parses the event data to form assessment data. The process then generates the risk assessment model using the assessment data.

Using the method and apparatus disclosed herein, a patient risk assessment model may be generated from a collection of dynamic data. The patient risk assessment model describes the morbidity and mortality factors contributing to a condition of a patient at a medical care facility. The morbidity and mortality factors may be derived from event data collected from a set of sensors in addition to the traditional static data elements, such as patient medical records and observations of medical care workers. The use of a more comprehensive source of data provides a more robust solution for providing a patient risk assessment. The patient risk assessment model may then be used to formulate a patient treatment strategy.

Additionally, the method and apparatus described above may be deployed in a variety of medical care facilities. For example, a set of sensors may be deployed in a hospital, nursing home, outpatient care facility, or in a user's residence. The event data gathered from the set of sensors may be collected into a central database for generating a patient risk assessment model for non-traditional settings.

The invention can take the form of an entirely hardware embodiment, an entirely software embodiment or an embodiment containing both hardware and software elements. In a preferred embodiment, the invention is implemented in software, which includes but is not limited to firmware, resident software, microcode, etc.

Furthermore, the invention can take the form of a computer program product accessible from a computer-usable or computer-readable medium providing program code for use by or in connection with a computer or any instruction execution system. For the purposes of this description, a computer-usable or computer-readable medium can be any tangible apparatus that can contain, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

Further, a computer storage medium may contain or store a computer readable program code such that when the computer readable program code is executed on a computer, the execution of this computer readable program code causes the computer to transmit another computer readable program code over a communications link. This communications link may use a medium that is, for example without limitation, physical or wireless.

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated.

What is claimed is:

1. A computer implemented method for generating a risk assessment model for a patient, the computer implemented method comprising:
retrieving event data for the patient, wherein the event data is derived from video data, and wherein the event data further comprises metadata describing events affecting the patient in a medical care environment;
parsing the event data to form assessment data; and
generating the risk assessment model using the assessment data.

2. The computer implemented method of claim 1, further comprising:
formulating a treatment strategy using the risk assessment model.

3. The computer implemented method of claim 1, wherein the event data describes events directly affecting a patient.

4. The computer implemented method of claim 1, wherein the event data describes events indirectly affecting a patient.

5. The computer implemented method of claim 1, wherein the risk assessment model comprises a set of patient morbidity factors.

6. The computer implemented method of claim 5, wherein the risk assessment model further comprises remedies for the set of patient morbidity factors.

7. The computer implemented method of claim 1, further comprising:
receiving the video data from a set of sensors associated with the healthcare facility; and
analyzing the video data to identify event data, wherein analyzing the video data comprises generating the metadata describing the events affecting the patient.

8. The computer implemented method of claim 7, wherein the set of sensors comprises a set of digital video cameras.

9. The computer implemented method of claim 1, wherein the risk assessment model is further generated using static data elements.

10. The computer implemented method of claim 1, wherein parsing the event data further comprises:
processing the dynamic data using at least one of a statistical method, a data mining method, a causal model, a mathematical model, a marketing model, a behavioral model, a psychological model, a sociological model, or a simulation model.

11. A computer program product comprising:
computer usable program code for generating a risk assessment model for a patient, the computer program product comprising:
computer usable program code for retrieving event data for the patient, wherein the event data is derived from video data, and wherein the event data further comprises metadata describing events affecting the patient in a medical care facility;
computer usable program code for parsing the event data to form assessment data; and
computer usable program code for generating the risk assessment model using the assessment data.

12. The computer program product of claim 11, further comprising:
computer usable program code for formulating a treatment strategy using the risk assessment model.

13. The computer program product of claim 11, wherein the event data describes events directly affecting a patient.

14. The computer program product of claim 11, wherein the event data describes events indirectly affecting a patient.

15. The computer program product of claim 11, wherein the risk assessment model comprises a set of patient morbidity factors.

16. The computer program product of claim 15, wherein the risk assessment model further comprises remedies for the set of patient morbidity factors.

17. The computer program product of claim 11, further comprising:
computer usable program code for receiving the video data from a set of sensors associated with the medical care facility; and
computer usable program code for analyzing the video data to identify event data, wherein analyzing the video data comprises generating the metadata describing the events affecting the patient.

18. The computer program product of claim 11, wherein the risk assessment model is further generated using static data elements.

19. The computer program product of claim 12, wherein the computer usable program code for parsing the event data further comprises:
computer usable program code for processing the event data using at least one of a statistical method, a data mining method, a causal model, a mathematical model, a marketing model, a behavioral model, a psychological model, a sociological model, or a simulation model.

20. A system for creating a risk assessment model for a patient, the system comprising:
a database, wherein the database stores event data collected by a set of sensors; and
an analysis server, wherein the analysis server retrieves event data for the patient, wherein the event data is derived from video data, and wherein the event data further comprises metadata describing events affecting the patient in a medical care environment; parses the event data to form assessment data; and generates the risk assessment model using the assessment data.
